(19) United States
(12) Patent Application Publication
(10) Pub. No.: US 2004/0163154 A1
(43) Pub. Date: Aug. 26, 2004

(54) Pitcher's jacket

(76) Inventor: Bradley Cooper, Sugar Land, TX (US)

Correspondence Address: Bradley Cooper, 7323 Emerald Glen Dr., Sugar Land, TX, 77479, US

(21) Appl. No.: 10/783,119

(22) Filed: Feb. 20, 2004

Related U.S. Application Data
(60) Provisional application No. 60/448,448, filed on Feb. 21, 2003.

(51) Int. Cl. A61F 9/00

(52) U.S. Cl. 2/69

(57) ABSTRACT

A jacket for a person engaged in a pitching activity is described herein for retaining warmth in the arm and upper extremity muscles used in pitching. It is desirable for pitchers to retain warmth in the favored arm and upper extremity muscles, in order that these muscles remain loose and flexible to prevent cramping and tightening. The jacket includes a sleeve and upper extremity portion proximate the sleeve for the pitching arm, while the non-pitching arm and the associated upper extremity portion are not covered by the jacket. The jacket includes a portion covering the upper extremity of the back that surrounds the body and helps maintain the position of the jacket during activity. A fastener element is provided to permit the jacket to be engaged and separated from the wearer. The jacket provides coverage and warmth without restricting motion and, therefore, is designed to allow freedom of movement for the athlete while actively engaged in activity or when idle awaiting return to play.

This application claims priority based on Provisional Patent Application No. 60/448,448 filed on Feb. 21, 2003.

### BACKGROUND OF THE INVENTION

1. Field of the Invention

This invention relates generally to sports apparel, and more particularly, to a jacket useful for maintaining warmth for a participant's pitching arm.

2. Background of the Invention:

In the field of athletics, it is standard practice for athletes to try to maintain warmth in the particular muscle groups being used in the activity. In the case of sports with overhead pitching activity such as baseball or softball, the pitcher tries to maintain muscle warmth in the arm and upper extremity muscle groups of the involved, pitching arm. The muscles utilized in pitching include those in the arm and elbow region, specifically the Flexor/Pronator mass, Biceps, and Triceps, as well as the shoulder and back region which includes the Deltopectoral, Rotator Cuff, and Scapulothoracic muscles. Warmth retention keeps the muscles loose and flexible, thereby reducing muscle cramps and tightening. Since it is widely recognized that warm muscles generally perform better, athletes typically “warm up” prior to play. Although the muscles and tissue are warmed up by use, athletic activity is often discontinued for short periods of time throughout the course of play. It is important that the pitcher not only maintain muscle warmth and readiness during activity, but also while waiting to return to play.

Typical solutions for maintaining muscle warmth in the pitching arm and upper extremity include, wearing a jacket or wrapping towels around the arm. In the case of a jacket; the pitcher inserts their pitching arm into the sleeve of a jacket, while allowing the remainder of the jacket to hang down off the body of the wearer. The free sleeve and remainder of the jacket interfere with the player's ability to move properly during play. In addition, the player often experiences discomfort and an increased risk of over-heating while performing in warm climates since a full jacket does not allow for heat dispersion from the uninvolved areas. In the case of wrapping towels around the arm, the pitcher simply drapes a towel around their arm Difficulties have been encountered when using this solution since there is no mechanism to keep the towel attached and the muscles in the shoulder and upper extremity area are not addressed in this solution.

Attempts have been made to provide devices that cover only the arm of the pitcher. Known prior art disclosing coverage of the arm only include U.S. Pat. No. 5,683,546 to Vita, U.S. Pat. No. 359,835 to Hadfield, U.S. Pat. No. 5,909,801 to Coffman, U.S. Pat. No. 6,052,824 to May. These devices disclose thermal benefit to the arm and do not address the other muscle groups in the upper extremities that are used in pitching.

Still other devices such as U.S. Pat. No. 4,569,087 to Kerwin and U.S. Pat. No. 4,985,934 to Perry disclose a garment comprising a pocket for the receipt of a therapeutic medium such as a cold pack. U.S. Pat. No. 4,985,934 also discloses a set of straps, which have a tendency to bind and restrict movement.

### SUMMARY OF THE INVENTION

An object of the invention is to provide a jacket for pitchers that allows for normal heat dispersion from the other, uninvolved areas of the body.

Still another object of the present invention is to provide a jacket for pitchers that can be worn continuously during the course of play, while idle, and/or during warm up and cool down activities.

A further object of the present invention is to provide a jacket for pitcher's that does not unduly restrict the movements of the wearer and allows freedom of movement while at play or at rest.

Another object of the present invention is to provide a jacket for a pitcher that is of unitary design, without removable straps or appendages.

The foregoing and other objects are accomplished, according to the present invention by providing a jacket for pitchers, which provides coverage and enables retention of warmth in the involved arm and upper extremity muscles used for throwing. Specifically, thermal benefit is provided for the arm muscles of the Flexor/Pronator mass, Biceps, and Triceps, as well as, the Deltopectoral, Rotator Cuff, and Scapulothoracic of the shoulder and back muscles.

It is a still further object of the present invention to provide an outerwear garment for pitchers that secures to the wearer's body with a single fastener element. This fastener element can be easily released to permit convenient positioning and removal of the garment.

It is also an object of the present invention to provide an outerwear garment for pitcher's that can be worn over the player's uniform and allows for team and player identification on the garment.

The above and further objects, details and advantages of the present invention will become apparent from the following detailed descriptions, when read in conjunction with the accompanying drawings.

### BRIEF DESCRIPTION OF THE DRAWINGS

FIG. 1 is a front view of the outerwear garment for pitchers embodying the present invention.

FIG. 2 is a front view of a typical athlete wearing the outerwear garment for pitchers.

FIG. 3 is a front view, illustrating the garment being secured to the wearer by means of the fastener element.

FIG. 4 is a rear view of a person wearing the garment, illustrating how the garment appears when viewed from the rear.

It should be observed that the elements and operation of the embodiment of the present invention have been illustrated in somewhat simplified form in each of the drawings and in the following specification in order to eliminate unnecessary details which would be apparent to someone skilled in this art. Therefore, other specific forms and constructions of the invention will be equivalent to the embodiments described although departing somewhat from the exact appearance of the drawings.

### DETAILED DESCRIPTION OF THE INVENTION/FIGURES

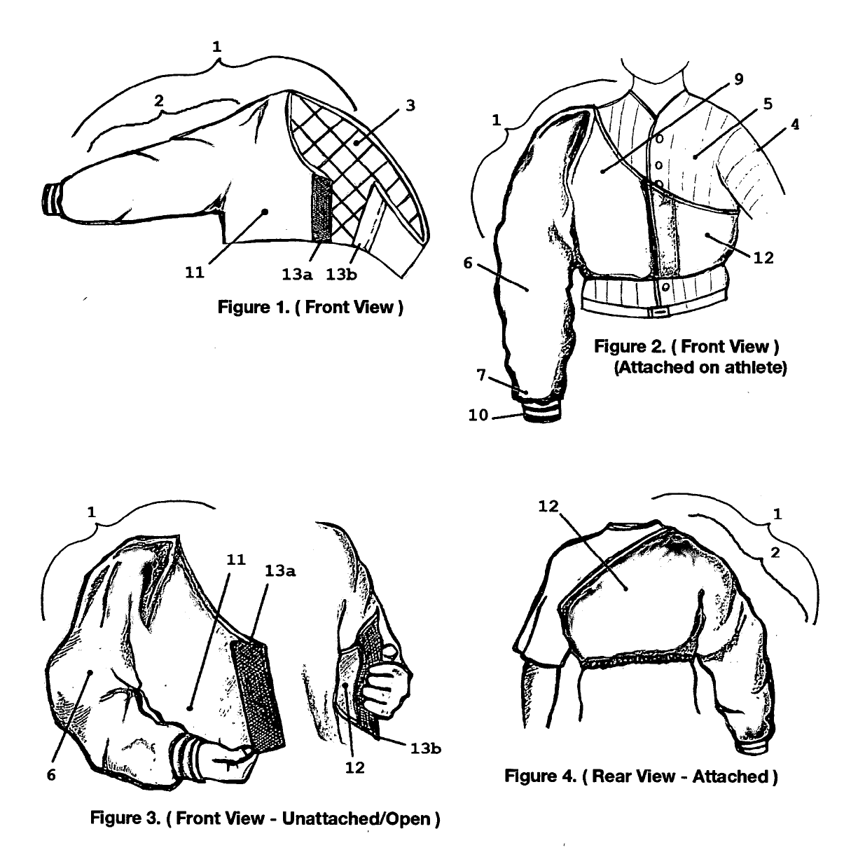

The novel outerwear garment for pitchers is illustrated generally as 1 in FIGS. 1,2, 3 and 4. In FIG. 1, the garment 1 is illustrated as having an outer shell of fabric 2 that encompasses the entire garment 1. The outer shell of fabric 2 is also viewed from the rear as illustrated in FIG. 4. This outer shell fabric 2 provides a non-porous coverage barrier and inhibits the passage of ambient air. Also indicated in FIG. 1, the garment 1 includes an additional layer of insulative fabric that forms the inner lining 3. The inner lining 3 is attached to the inner wall of the outer shell 2 to insulate the heat radiating from the arm and upper extremity region of the wearer. In FIG. 2 the garment 1 is being illustrated as being worn by a typical athlete 4 over an athletic uniform 5. It can be seen that one arm of the athlete 4 is inserted through a tubular sleeve 6 which includes a wrist end 7 and a shoulder end 8. The shoulder end 8 is attached at one end to a body portion 9 that is partially wrapped about the upper extremity of the wearer. Sleeve 6 should be provided approximately in length equivalent to length of the wearer's arm and can be provided for a left or right-handed person. The sleeve 6 should be provided approximately in width equivalent to the wearer's arm with additional width to allow for freedom of movement in the arm and upper extremity. The sleeve 6 is integrally formed with the body portion 9 via a shoulder portion 8 so that unitary construction exists. Also it can be seen that the free end of the sleeve at the wrist end 7 is provided with a cuff 10 to prevent the loss of heated air from this end of the sleeve. The cuff 10 may take the form of a webbed construction that forms a loose seal and elastically closes about the wrist of the wearer.

FIG. 2 further illustrates that the body portion of the garment, represented in general by numeral 9, includes a front area identified by numeral 11 and a back area identified as numeral 12. The back area 12 of the body portion 9 is further illustrated in FIG. 4. The back area 12 of the body portion 9 covers the scapulothoracic portion of the back and ends with a fastener element 13b. The front area 11 of body portion 9 covers the upper chest quadrant of the favored upper extremity and ends at the center of the chest with the fastener element 13a. Once the athlete inserts his/her arm into the sleeve 6, the athlete brings the end of the back area 12 underneath the opposite arm and brings it across the front of the chest. As seen in FIG. 3, the athlete then secures the garment by attaching the fastener element 13b at the free end of the back area 12 onto the fastener element 13a at the free end of front body area 11. In the first preferred embodiment, the fastener element indicated as 13a comprise a plurality of hooks, which detachably engage with the plurality of loops in fastener element 13b. As illustrated in FIG. 2, once the athlete secures fastener element 13a to fastener element 13b, the fastener elements are discreetly concealed.

Many modifications and other embodiments of the invention will come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A jacket comprising;
a portion of the jacket surrounding the upper extremity below the arms; and a portion of the jacket extending over the front chest quadrant, shoulder, and at least a portion of a first arm, wherein a second arm and associated upper extremity and shoulder region are not covered by the jacket.

2. The jacket recited in claim 1 wherein the arm covered by the jacket is the arm used in pitching.

3. The jacket as recited in claim 2 further comprising a fastener element, the fastener element permitting the region portion of the jacket surrounding the chest to be separated.

4. A method for providing a warm-up jacket, the jacket retaining warmth for one arm and associated muscles, the method comprising:
covering the one arm by the jacket;
shaping the jacket to expose the second arm and associated abdominal region; and
providing a lower portion of the jacket to secure the jacket to the abdomen.

5. The method as recited in claim 4 wherein the one arm is involved in a throwing activity.

6. The method recited in claim 4 further comprising providing the lower portion with a detachable fastener to permit removal of the jacket.

7. A jacket comprising;
a portion covering at least a portion of one arm and associated regions of the abdomen;
an aperture in the jacket exposing a second arm and associated regions of the abdomen; and
a lower portion securing the jacket to the abdomen.

8. The jacket as recited in claim 7 wherein the lower portion includes a fastener for detachably securing the jacket to the abdomen.

9. The jacket as recited in claim 7 wherein the jacket permits the one arm and associated regions to retain heat.
